(19) United States
(12) Patent Application Publication
(10) Pub. No.: US 2017/0128617 A1
(43) Pub. Date: May 11, 2017

(54) SEALANT FORMULATION AND USES THEREOF

(71) Applicant: Ethicon, Inc., Somerville, NJ (US)

(72) Inventors: Ashley DeAnglis, Skillman, NJ (US); Anne J. Gorman, Hightstown, NJ (US)

(21) Appl. No.: 15/345,722

(22) Filed: Nov. 8, 2016

Related U.S. Application Data
(60) Provisional application No. 62/253,950, filed on Nov. 11, 2015.

(51) Int. Cl. A61L 24/10; A61M 5/31; A61B 17/00; A61L 15/44; A61L 24/00

(52) U.S. Cl. CPC A61L 24/106; A61L 15/44; A61L 24/0015; A61B 17/00491; A61M 5/3129; A61L 2400/04; A61L 2300/418

(57) ABSTRACT

The present invention relates to a sealant formulation comprising a blend containing fibrinogen and an exogenous activator of a member of a mammalian blood clotting cascade.

### TECHNOLOGICAL FIELD

The present disclosure concerns fibrinogen based sealant formulations and uses thereof.

### BACKGROUND ART

References considered to be relevant as background to the presently disclosed subject matter are listed below:
U.S. Pat. No. 8,962,033

Acknowledgement of the above references herein is not to be inferred as meaning that these are in any way relevant to the patentability of the presently disclosed subject matter.

### BACKGROUND

Fibrin sealant formulations for hemostasis typically contain two liquid components, fibrinogen and thrombin, which are stored frozen or in the lyophilized state.

U.S. Pat. No. 8,962,033 describes a fibrin matrix and specifically a method for applying fibrin matrix onto a leaking tissue, the method comprising applying an amount of a solid fibrin sealant blend onto the tissue, the solid blend comprising a proteolytic enzyme capable of forming fibrin when it reacts with fibrinogen, and then applying onto the solid fibrin sealant an amount of a liquid fibrin sealant formulation. The combination of the solid fibrin and the liquid fibrin form a fibrin matrix on the tissue.

### General Description

The present disclosure is based on the development of a sealant formulation that is stable when stored in liquid form. Specifically, and in accordance with a first of its aspects, the present disclosure provides a sealant formulation comprising a blend containing fibrinogen and an activator of a member of a mammalian blood clotting cascade, wherein said activator is exogenous to said mammalian cascade and said member is upstream to fibrinogen in the mammalian blood clotting cascade.

In some embodiments, the sealant formulation is a liquid blend. The liquid blend was found to be stable at 2° C.-8° C. as well as at room temperature for at least 5 days.

In some embodiments, the activator comprises venom, a venom component or an analog of a venom component. Examples for venom or venom components include, without being limited thereto, Ecarin, Russell's Viper venom X factor (RVV-X), Russell's Viper venom V factor (RVV-V), Noscarin, Oscutarin, Trocarin, Convulxin, Botrocetin and any combination of said venom component.

In some embodiments, the activator is Ecarin.

In some embodiments, the activator is RVV-X.

In some embodiments, the activator is RVV-V.

In some embodiments, the activator is a combination of RVV-X and RVV-V.

The present disclosure also provides an applicator comprising a barrel holding the sealant formulation disclosed herein; and a re-sealable opening for delivery therethrough of the sealant formulation upon need.

In some embodiments, the applicator is in a form of a syringe holding the sealant formulation in liquid form.

In accordance with yet another aspect, the present disclosure provides a wound dressing comprising a support matrix holding the sealant formulation disclosed herein.

In accordance with a further aspect, the present disclosure provides a method for promoting the formation of a fibrin clot, the method comprises contacting blood with the sealant formulation disclosed.

In accordance with yet another aspect, the present disclosure provides a method of treating a bleeding wound in a subject in need thereof, the method comprises applying onto at least a portion of said wound an amount of the sealant formulation disclosed herein, the amount of said sealant formulation is effective to promote clotting in the subject's wound.

Finally, provided herein, in accordance with another aspect, is a kit comprising a sealant formulation as disclosed herein, and instructions for use of the sealant formulation to promote clotting in said wound, said instructions comprises applying the sealant formulation onto at least a portion of said wound.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a graph showing the clotting time as a function of Ecarin concentration.

FIG. 2 is a graph showing the stability of clotting time after storage of a sealant formulation at room temperature or at in 2° C.-8° C., the stability being a function of time after Ecarin was added (minutes).

FIG. 3 is a graph showing the stability of clotting time after storage of a sealant formulation at room temperature or at 2° C.-8° C., the stability being a function of time after Ecarin was added (days).

FIG. 4 is a graph showing the average clotting time as a function of RVV-X concentration with or without RVV-V.

FIG. 5 is a graph showing log of the average clotting time as a function of RVV-X concentration with or without RVV-V.

FIG. 6 is a graph showing the average of clotting time (s) at room temperature or at storage in 2° C.-8° C. as a function of time after RVV-X was added (minutes).

FIG. 7 is a graph showing the stability of clotting time at room temperature or at storage in 2° C.-8° C. as a function of time after RVV-X was added (days).

### DETAILED DESCRIPTION OF EMBODIMENTS

The present invention is based on the development of a ready to use, All-In-One (AIO) sealant formulation. The inventors have developed a formulation that can be delivered in a syringe (single barrel) without clogging (e.g. from undesired clotting), a phenomena prevailing with currently available sealants.

Specifically, it has been found that by combining fibrinogen with an activator of one of the critical members in the clotting cascade, i.e. an activator that does not directly act on fibrinogen, the combined blend is stable, even in liquid form. This formulation would be liquid stable since the formulation does not contain any of the enzyme precursors needed for clotting, e.g, prothrombin, factor X, etc. In the presence of blood, which contains the member, e.g. zymogens, clotting would be rapidly activated by the activator.

As shown in the non-limiting Examples, the use of activators that act on members of the clotting cascade upstream to fibrinogen, drastically reduced clotting times with plasma to less than 30 seconds compared with more than 490 seconds without the activator. The mixture of the activator with fibrinogen was found to be stable for at least 5 days even at room temperature (herein defined as a temperature equal or between 20-25° C.) and unaffected by freezing and thawing.

Thus, in accordance with a first aspect, there is provided herein a sealant formulation comprising a blend containing fibrinogen and an activator of a member of a mammalian blood clotting cascade, wherein said activator is exogenous to said mammalian cascade and said member is upstream to said fibrinogen in the blood clotting cascade.

In the context of the present invention, the term “sealant formulation” is to be understood as a tissue adhesive, the formulation having ingredients that upon contact with the tissue or blood react to subsequently form into a tissue adhesive and thereby stop bleeding and/or seal physiological leaks, e.g, of cerebrospinal fluids (CSF), lymph, bile, gastrointestinal (GI) content, air leak from lungs etc. In the context of the present disclosure it is to be understood the that formulation ingredients do not interact with each other and are essentially inert until they are brought into contact with the member upon which the activator acts. Once the member is activated by the activator, clotting initiates and fibrin clot based tissue adhesive is formed.

The sealant formulation comprises a blend of at least fibrinogen and the activator. When referring to a “blend” it is to be understood as any form of a mixture, homogenous and non-homogenous mixture of at least the fibrinogen and the activator. The blend may include other ingredients as further detailed below. However, the formulation is free (i.e. absent) of the member of the clotting cascade that is activated by the activator or of any active member of the clotting cascade that is downstream to said activator. In other words, the formulation is designed to include only components of the blood clotting cascade that do not interact with each other. As such, the formulation does not include thrombin as this would interact with fibrinogen.

In the context of the present disclosure the blend is a liquid blend. When referring to a liquid blend it is to be understood as one being liquid at room temperature. To this extend, the present disclosure also encompasses sealant formulations in frozen form such that upon use, the product is thawed to be, at room temperature, in its liquid to state.

The formulation comprises at least two components that promote the formation of blood clotting, one being the fibrinogen and the other being the activator.

In the context of the present invention, the activator is a biological or chemical entity that upon contact with blood, causing the member to undergo a biochemical change into its active form.

In some embodiments, the activator is exogenous to the mammalian, in particularly to the mammalian's blood clotting cascade. In this context, it is to be understood that an exogenous activator refers to a material (such as a protein) that is capable of activating a coagulation cascade of a mammalian species, but is not normally present in that particular mammalian species. For example, and without being limited thereto, the snake venom enzyme Ecarin, which is not normally present in the blood of mammals, is an activator of a member of the human blood clotting cascade. Thus, when using the term “exogenous” it is for the mere purpose of distinguishing the activator from a component normally contained within the mammalian clotting cascade.

In some embodiments, the activator is a proteolytic enzyme having activity upstream to said member of the mammalian clotting cascade (but, being exogenous to the particular mammalian cascade, i.e. does not naturally participate in the particular mammalian clotting cascade).

In some embodiments, the activator is a protease.

In some embodiments, the protease is a serine protease.

In some other embodiments, the protease is a metalloprotease.

In some embodiments, the activator is snake venom or comprises a component such as a protease from snake venom, or is an analog of a venom component, such snake venom or analog are inherently exogenous to mammalian species. An analog can be, for example, a recombinant form of the venom component or a modified form thereof, as further discussed below. The analog, whether recombinant or modified is one that maintains the functionality/activity of participating in the clotting cascade such that when the sealant formulation is brought into contact with blood, the analog activates a member of the blood clotting cascade to the extent that a sealant comprising fibrin clot is formed.

In some embodiments, the activator activates prothrombin. The activator may be, for example one or the combination of: Ecarin, isolated from the venom of the saw-scaled viper (Echis carinatus); Noscarin extracted from the venom of the Australian tiger snake (Notechis scutatus scutatus); Trocarin extracted from the rough scaled snake Tropidechis carinatus; Oscutarin extracted from the venom of the Taipan snake Oxyuranus scutellatus. Each of said activators are to be considered independent embodiments of the present disclosure.

In some embodiments, the activator is RVV factor X activator (RVV-X) from Russell's viper venom that activates factor X.

In some embodiments, the activator is RVV-factor V activator (RVV-V) also from Russell's viper venom (D. russelii) that activates factor V;

In some embodiments, the activator acts on platelets and is any one or combination of Convulxin that activates platelets; Botrocetin isolated from Bothrops jararaca causing platelet aggregation; Ristocetin isolated from Amycolatopsis lurida causing platelet aggregation.

In yet some embodiments, the activator is one that does not increase, directly or indirectly, the rate of clotting enzyme generation or activation, e.g. by the induction of platelet aggregation.

In some embodiments the activator is a recombinant form of a coagulation enzyme. Examples of recombinant venom prothrombin activators are describes, inter alia, by Ann Lovgren (“Recombinant snake venom prothrombin activators” Bioengineered 4:3 153-157 2013, the content of which is incorporated herein in its entirety). These include, inter alia, ecarin, trocarin, oscutarin

In some embodiments, the activator comprises or is Ecarin.

In some embodiments, the activator comprises or is RVV-X.

In some embodiments, the activator comprises or is RVV-V.

In some embodiments, the activator is exogenous to mammalian species, e.g. is of non-human source, it is, at times, modified with substances that mask the activator from the mammalian host's immune response. Thus, in the context of the present disclosure, when referring to an activator, it is understood to include also an activator per se (i.e. the active entity per se) that has been modified in order to improve the performance of the activator. The improvement in performance can be by increasing circulation time of the activator in the blood, reduce immune response against the activator and the like. Without being limited thereto, such a modified activator can comprise the activator per se and a substance selected from the group consisting of polyethylene glycol (PEG), carbohydrates, or polysaccharides, such as dextran.

In some embodiments, the activator is modified with PEG. i.e. the activator is pegylated. In this connection, when referring to an activator, it is to be understood as referring also to the modified activator.

In the context of the present disclosure each of the above listed activators, their recombinant forms or functional analogs are to be considered individual and separate embodiments of the present disclosure even if recited as a group of activators.

As defined herein, the activator activates a member of a mammalian clotting cascade.

In the context of the present disclosure the “member of a/the clotting cascade” is to be understood as referring to any biological substance that participates in the blood clotting cascade. The member should preferably be a critical participant in the cascade i.e. that in its absence, no clotting or insufficient clotting takes place and no or insufficient fibrin matrix is formed.

In some embodiments, the member is a zymogen. As appreciated, a zymogen is an inactive enzyme precursor and requires a biochemical change (such as a hydrolysis reaction revealing the active site, or changing the confirmation to reveal the active site) for it to become an active enzyme.

In some embodiments, the zymogen is a precursor of a serine proteinase. In some further embodiments, the zymogen is any one of prothrombin, Factor X, Factor IX, Factor XI, Factor XII, Factor VII.

In one embodiment, the zymogen is prothrombin.

In some embodiments, the member is a factor or co-factor participating in the pathway of the clotting cascade. In some embodiments, a factor or co-factor member of the clotting cascade is selected, without being limited thereto, from the group consisting of Factor V and Factor VIII. Factor V and Factor VIII are co-factors in the coagulation cascade that accelerate clotting reactions.

In some embodiments, the member is platelet. In some embodiments, when the member comprises platelets, the activator is one selected from the group consisting of

### Convulxin; Botrocetin; Ristocetin.

The amounts of the fibrinogen and of the activator can vary. In some embodiments, the amounts are such that the molar ratio between the two components, i.e. between fibrinogen and the activator is in the range of 70,000:1 and 25:1. This range is based, inter alia, on the lowest activator (in example, Ecarin) tested amount that was found to be effective in causing clotting, said lowest amount providing a fibrinogen to activator ratio of 66,094:1; and the highest tested amount of activator that showed to be effective, providing a ratio of up to about 26:1.

The formulation can comprise other ingredients required for the promotion of a clot. In some embodiments, the sealant formulation comprises calcium. For example, as shown in the following non-limiting examples, RVV-X requires the presence of calcium ions in order to activate factor X.

In some embodiments, the concentration of the activator is at least 3 mg/ml, at times, at least 4 mg/ml, 5 mg/ml, 6 mg/ml, 7 mg/ml, 8 mg/ml, 9 mg/ml, 10 mg/ml and at most 100 mg/ml, at times, at most 95 mg/ml, 90 mg/ml, 85 mg/ml, 80 mg/ml, 75 mg/ml, 70 mg/ml, 65 mg/ml, 60 mg/ml, 55 mg/ml, 50 mg/ml, 45 mg/ml, 40 mg/ml, 35 mg/ml, 30 mg/ml, 25 mg/ml, 20 mg/ml, 15 mg/ml, 10 mg/ml.

When calcium is part of the formulation, its concentration can vary. In some embodiments, the calcium concentration in the formulation is at least 3 mg/ml, 4 mg/ml 5 mg/ml, 6 mg/ml, 7 mg/ml or 8 mg/ml and at most 20 mg/ml, 19 mg/ml 5 mg/ml, 18 mg/ml, 17 mg/ml, 16 mg/ml, 15 mg/ml, 14 mg/ml, 13 mg/ml, 12 mg/ml, 11 mg/ml, 10 mg/ml or 9 mg/ml.

In some embodiments, the sealant formulation comprises other components which can assist in the performance of the sealant formulation. For example, and without being limited thereto, the sealant formulation comprises one or more of factor XIII, anti-fibrinolytic agents (such as aminocaproic acid (ε-aminocaproic acid), aprotinin and tranexamic acid) antibiotics, stabilizers such as arginine, lysine, fibronectin, von Willebrand factor; RGD peptides; growth factors, cartilage inducing factors, osteoid inducing factors, bone growth factors, collagen growth factors; cytokines; interferons; hormones; therapeutic agents such as antimicrobial agents, anti-inflammatories; anti-cancer drugs; chemotherapy agents; analgesics; interleukins; minerals; molecules which stimulate cell migration, adhesion and/or proliferation; enzymes; neurotrophic factors such as nerve growth factor (NGF); ciliary neurotrophic factor (CNTF), their pharmaceutically acceptable salts, or mixtures thereof, etc, such additives being selected as known to those versed in the art of sealant formulations. A list of possible sealant additives can be found, inter alia, in U.S. Pat. No. 8,858,969 to Z-Medica “Hemostatic Compositions, Devices and Methods”; and US patent application publication No. 2014/0271610 to Orthovita “Gelatin and Alginate-Based Formulations for Hemostasis”; the contents of which are incorporated herein by reference.

The sealant additives can be isolated from plasma of human beings or mammals or can be recombinant.

Fibrinogen is naturally a blood-derived soluble protein which can be purified from blood plasma and is converted to a fibrin monomer e.g. by the enzyme thrombin. Fibrin monomer spontaneously assembles into a fibrin polymer that makes up a blood clot.

The fibrinogen component can be prepared from initial blood composition. The blood composition can be whole blood or blood fractions, i.e. a product of whole blood such as plasma. Fibrinogen can be autologous, human including pooled plasma, or of non-human source. It is also possible that the fibrinogen is prepared by recombinant methods or can be chemically modified.

In the context of the present disclosure, when referring to fibrinogen it is to be understood as referring to purified and isolated fibrinogen per se but also to biologically active component (BAC) of blood plasma comprising fibrinogen concentrated viral-inactivated cryoprecipitate of human plasma comprising solution of blood plasma derived proteins.

In some embodiments, the fibrinogen is provided as part of the biologically active component (BAC). There are several types of BAC. In some embodiments, BAC is a biologically active component that contains tranexamic acid, as an antifibrinolytic agent. BAC containing tranexamic acid is sometimes known by the product name Quixil (Omrix, Israel).

In some other embodiments, BAC is a biologically active component that does not contain tranexamic acid. This is considered a second generation BAC and is referred to in the art as BAC2. BAC2 is considered a stable form of BAC where during its preparation, plasminogen (the enzyme precursor of plasmin, which breaks down fibrinogen and fibrin) is removed.

In a preferred embodiment, BAC is BAC2, i.e. a biologically active component that lacks tranexamic acid. BAC2 is typically prepared as component A in the disclosure of EP 534 178. For example, component A therein is prepared from concentrated cryoprecipitate, and undergoes viral inaction by solvent detergent treatment and pasteurization. In some embodiments, BAC2 is a concentrated viral inactivated cryoprecipitate comprising mainly fibrinogen and is plasminogen-depleted (the removal of plasminogen can be carried out as described in EP 1 390 485). The BAC2 is without anti-fibrinolytic agents.

In some embodiments, the fibrinogen is a biologically active component of a cryoprecipitate-derived antihaemophilic factor preparation. As known in the art, antihemophilic factor is also known as Factor VIII concentrate, which is also rich in fibrinogen.

Any other source of fibrinogen can be used as long as the fibrinogen source does not contain a zymogen (inactive precursor) to the member onto which the exogenous activator acts.

Examples of fibrinogen sources include, but are not limited to, recombinant fibrinogen, purified fibrinogen, including fibrinogen component of EVICEL (i.e, BAC2), fibrinogen component of Tisseel (containing aprotinin, an antifibrinolytic agent.

The BAC solution can further comprise stabilizers such as arginine, lysine and other sealant additives as known in the art.

In some embodiments, BAC and preferably BAC2 can be derived from cryoprecipitate (Cryoprecipitate-derived Antihaemophilic Factor which is a frozen blood product prepared from plasma as explained below), in particular concentrated cryoprecipitate.

Herein, unless otherwise stated, when referring to BAC it is to be understood as preferably, but not exclusively, referring to BAC2.

Specifically, and in the context of the present disclosure, the term “cryoprecipitate” refers to a blood component which is obtained from frozen plasma prepared from whole blood. A cryoprecipitate can be obtained when frozen plasma is thawed in the cold, typically at a temperature of 0-4° C., resulting in the formation of precipitate that contains predominantly fibrinogen. The precipitate can be collected, for example by centrifugation and dissolved in a suitable buffer such as a buffer containing 120 mM sodium chloride, 10 mM trisodium citrate, 120 mM glycine, 95 mM arginine hydrochloride. The solution of BAC can comprise additional factors such as for example factor XIII, factor VIII, fibronectin, von Willebrand factor (vWF), vitronectin, etc. BAC can be prepared as described in U.S. Pat. No. 6,121,232 and/or WO98/033533 the contents of which is incorporated by reference. The composition of BAC can comprise stabilizers such as anti-fibrinolytic agents (e.g. tranexamic acid) and arginine hydrochloride. The amount of anti-fibrinolytic agents such as tranexamic acid in the BAC can be from about 80 to about 110 mg/ml.

In some embodiments, the concentration of plasminogen and/or plasmin in the solution derived from the blood to obtain the BAC is actively lowered to be equal or less than 15 μg/ml, at times, equal or less than 12 μg/ml, 10 μg/ml or even 5 μg/ml or less plasminogen. Lowering of the plasminogen and/or plasmin can be achieved according to the method described in any one of U.S. Pat. No. 7,125,569, EP 1,390,485 and WO02/095019 the content of each being incorporated herein by reference.

In some embodiments, the BAC contains plasminogen and/or plasmin. When these are present, an anti-fibrinolytic agent e.g. tranexamic acid or aprotinin is required. However, when the concentration of plasminogen and plasmin is lowered to be equal or below 15 μg/ml, the BAC (e.g. BAC2) can be free of anti-fibrinolytic agents (as the concentration of plasmin would be ineffective to cause fibrinogen degradation).

The fibrinogen can also be one that is commercially available, such as the BAC2 component (fibrinogen component from EVICEL®), as noted above, or any other fibrinogen containing solution, such as purified fibrinogen, recombinant fibrinogen or cryoprecipitate produced from human plasma. In this context, BAC2 is known as a frozen sterile solution pH 6.7-7.2, which consists mainly of a concentrate of human fibrinogen (55-85 mg/ml) and other ingredients being arginine hydrochloride, glycine, sodium chloride, sodium citrate, calcium chloride, water for injection (WFI).

An essential feature of the sealant formulation is that it is free of thrombin or thrombin-like molecules (i.e. molecules that have the same biological functionality as thrombin in the blood clotting cascade).

The sealant formulation can be in any physical form. In some embodiments, the formulation is in dry form, such as lyophilized formulation.

In some other embodiments, the sealant formulation is in liquid form, i.e. a liquid blend of at least the activator and fibrinogen. When in liquid form, the liquid carrier is typically a buffer to maintain the pH of the sealant formulation near neutral, e.g. pH 7.0±0.5.

Notwithstanding the physical phase of the formulation (e.g. liquid, lyophilized etc.) the formulation is stable under storage conditions. Stability is determined as absent of fibrin clots in the formulation after storage at 25° C. for at least 5 days. The stability is also apparent from the lack of visible aggregations during the storage, particularly when the formulation is in liquid form. In addition and in accordance with the present disclosure, when referring to a stable sealant formulation it is also to be understood as one that, upon use, has clotting time that is not affected by its storage temperature. In other words, upon use, the sealant formulation will clot at essentially the same time period irrespective of whether it was stored at room temperature or at lower temperatures, e.g. between 1° C. to room temperature, or 2° C.-8° C.

The present disclosure also provides an applicator for delivery of the sealant formulation. In the context of the present disclosure, the applicator is a device.

In some embodiments, the applicator comprises a barrel holding the sealant formulation disclosed herein and a re-sealable opening for delivery therethrough of the formulation.

In some embodiments, the applicator is a syringe holding in the syringe's barrel the formulation. The formulation may be in liquid form or in dry form, the latter is to be wetted (e.g. with saline) prior to application.

In some other embodiments, the applicator is in the form of a sprayer configured to apply the formulation by spraying.

The applicator can be for single use, i.e. to be disposed after all or a portion of the sealant formulation is used; or it may be designed for multiple uses such that the applicator's opening is resealed between uses.

In some other embodiments, the applicator is in a form of a wound dressing a support matrix holding a sealant formulation. In the context of this embodiment, the support matrix can be a non-woven fabric, such as those used in wet wipes onto which the formulation is absorbed, impregnated, swelled or the like. The support matrix with the sealant formulation can be contained within a container, such as in sachets, each being opened prior to use.

The present disclosure also provides a method of promoting the formation of fibrin matrix. According to the method, an effective amount of the sealant formulation is brought into contact with blood or blood plasma.

The present disclosure also provides a method of treating a wound, the method comprises applying onto at least a portion of said wound an effective amount of sealant formulation.

The application or contacting can be by any applicable means, including, without being limited thereto, mixing with the blood, spraying onto the bleeding wound, spreading onto the bleeding wound, dripping the formulation onto the bleeding wound, etc.

In the context of the present disclosure the “effective amount” is an amount sufficient to promote the formation of a fibrin matrix onto the wound. In some embodiments, the amount is effective to at least inhibit bleeding of the wound, at times, to cease bleeding of the wound and reduce the time required to achieve hemostasis. The amount can depend on various parameters such as the level of bleeding, the dimensions (size of incision and depth) of the wound, age and condition of the subject having the bleeding wound, and other factors which can be recognized by the skilled in the art. For example, the formulation can be applied in a volume range of 0.01 to 10 ml/cm².

In some embodiments, the effective amount can be an amount that will produce a rapid fibrin clot, i.e. formation of fibrin clot in less than 61 seconds.

In some embodiments, when the activator is RVV-X, the effective amount is between about 0.001-0.003 U/ml, at times about 0.002 U/ml (U designating a unit); when the activator is Ecarin, the effective is between about 0.05-0.2 U/ml, at times about 0.1 U/ml.

It is noted that the units (U) of the various possible activators, e.g. venom enzymes, are determined differently (since they are acting on different substrates). For

Ecarin, RVV-X, RVV-V, the units are defined as listed below.

Ecarin unit: One unit will activate prothrombin to produce one unit of amidolytic activity at pH 8.4 at 37° C. One amidolytic unit will hydrolyze 1.0 μmole of N-p-tosyl-Gly-Pro-Arg-p-nitroanilide per min at pH 8.4 at 37° C.

RVV-X: One unit is the amount of RVV-X which generates one international unit of factor Xa from factor X.

RVV-V: One is the activity of RVV-V required for complete activation of the factor V contained in 1 ml of normal plasma.

Further, the present disclosure provides a package (kit) comprising:
a. the sealant formulation disclosed herein;
b. instructions for use of the sealant formulation for promoting the formation of a fibrin clot on a bleeding wound, said instructions comprises applying the sealant formulation onto said wound.

In some embodiments, the sealant formulation is in dry form and the instructions comprise the step of applying the formulation onto the wound in dry form (to be wetted by the blood) or the step of wetting the dry formulation prior to application onto the wound.

In some embodiments, the package also comprises a sealant applicator configured to facilitate the contacting of the sealant formulation with the wound.

In some embodiments, the package is provided with the sealant formulation already within the applicator; in some other embodiments, the sealant formulation is supplied in a container and is introduced into the applicator prior to use.

In some embodiments, the applicator is a syringe as described herein above. In yet some other embodiments, the applicator is in the form of a wound dressing with the formulation being held onto a support matrix, as also described hereinabove.

The sealant formulation can be applied onto/incoporated into bandages, foams, wound dressings, pads and/or matrices. The formulation can be released into/onto a desired location from different delivery agents such as bandages, wound dressing, pads, foams, sponge and/or matrices. The agents can be made of natural and/or synthetic materials. Examples of such materials include, but are not limited to, polymers, hydrogels, polyvinyl alcohol (PVA), polyethylene glycol (PEG), hyaluronic acid, chondroitin sulphate, gelatin, alginate, collagen matrices, carboxymethylcellulose, dextran, poly(2-hydroxyethylmethacrylate) [PHEMA], agar, oxidize regenerated cellulose (ORC), self assembled peptides [SAPS], poly(glycolic) acid, poly(lactic) acid, fibrin and combinations thereof. In some embodiments, the sealant formulation disclosed herein is applied by spraying or dripping onto the tissue in need of sealing. In some embodiments, e.g. if a large surface area needs to be sealed from bleeding, spraying would be commonly used; if bleeding is occurring from a confined area, e.g. after a biopsy, then dripping would be commonly used. Alternatively, spraying and dripping can be used in the same procedure.

The delivery mode of the sealant can be determined based on various considerations, such as, the type/extent of sealing needed, the type/dimensions of opening (requiring sealing) and other considerations as appreciated by the physician.

As used herein, the forms “a”, “an” and “the” include singular as well as plural references unless the context clearly dictates otherwise. For example, the term “an activator” includes one or more activators which are capable of activating one or more members of the clotting cascade.

Further, as used herein, the term “comprising” is intended to mean that the formulation includes the recited components, i.e. the fibrinogen and the (one or more) activators, but not excluding other elements, such as physiologically acceptable carriers and excipients as well as other active agents. The term “consisting essentially of” is used to define formulations which include the recited components but exclude other elements that may have an essential significance on the clotting cascade. “Consisting of” shall thus mean excluding more than trace elements of other elements. Embodiments defined by each of these transition terms are within the scope of this invention.

Further, all numerical values, e.g. when referring the amounts or ranges of the components constituting the formulation, are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### NON-LIMITING EXAMPLES

### Materials and Methods

### Materials:

FACT Plasma, factor analyzed plasma from George King Biomedical, product number 020-1, 1 mL vial
Ecarin, Prothrombin Activator from Echis carinatus, from Enzyme Research Laboratories, Catalog Number P116-01-50EU, 50 EU Vial
RVV-X, Factor X Activator from Vipera russelli, from Enzyme Research Laboratories, Catalog Number P121-07-50U, 50 U Vial
RVV-V, Factor V Activator from Vipera russelli, from Enzyme Research Laboratories, Catalog Number P121-03-1000U, 1000 U vial
BAC2 Fibrinogen from Omrix, component of Evicel Fibrin Sealant (Human)
Purified Fibrinogen, Enzyme Research Laboratories, Catalog Number Fib3 Fibrinogen Stock calcium solution at 200 mM prepared from calcium chloride dihydrate, ACS grade, 99+%, Alfa Aesar,
Stock tris buffer at 20 mM, pH=7.4 prepared from Tris(hydroxymethyl)-aminomethane, 99%, Alfa Aesar,

### Method:

Multiple in vitro studies were performed using the Diagnostica Stago STart4 coagulation analyzer to assess clotting rates with various doses of enzymes, requirements for calcium, stability and impact of freezing-thawing. The analyzer measures the rate of fibrin clot formulation based on the impedance of a stainless steel ball oscillating within a disposable cuvette.

Specifically, for the clotting studies described in the following examples, 100 μL of the sample (typically consisting of fibrinogen, calcium and activator) was added to a cuvette in the analyzer. To initiate the reaction, 100 μL of pooled normal plasma (PNP, a mixture of plasma from multiple donors, usually at least 20 donors, to minimize variations in the clotting factor levels for different donors) was added to the cuvette, and the rate of clot formation was measured as described above. All clotting assays were performed at 37° C.

### Results

### Example 1: Clotting without Exogenous Activator

The goal of this experiment was to establish a baseline for clotting time of fibrinogen, calcium and plasma without the activator so as to establish a baseline. The optimal calcium concentration was also determined.

Purified fibrinogen, commercially available from Enzyme Research Labs, was used for the various studies as a fibrinogen source.

As shown on Table 1, clotting times were as short as 494.0 sec with 10 mM calcium. At 15 mM and 20 mM calcium clotting times were 563.4 and 683.0 sec, respectively. At other calcium concentrations above 20 mM, a clotting time was not registered by the analyzer within 999 sec.

TABLE 1
Clotting Time without Exogenous Activator

| | Clotting time (s) | | |
|---|---|---|---|
| Final Ca concentration (mM) | Replicate 1 | Replicate 2 | Mean |
| 7.5 | 795.2 | >999* | >795.2 |
| 10 | 470.9 | 517.0 | 494.0 |
| 15 | 574.8 | 552.0 | 563.4 |
| 20 | 664.6 | 701.4 | 683.0 |
| 30 | >999* | >999* | >999* |
| 40 | >999* | >999* | >999* |

*Analyzer's maximum measurement time of 999 sec

### Example 2: Ecarin Effect on Clotting

### 2A—Ecarin Dose Response

The goal of this study was to assess the impact on clotting time of various Ecarin concentrations, and determine a suitable concentration for future studies

Different concentrations of Ecarin ranging from 0-5 ecarin units per ml (see Table 2) were tested with fibrinogen (10 mg/mL), calcium (10 mM final concentration), and plasma. The fibrinogen/calcium/ecarin samples were warmed in the incubation wells of the coagulation analyzer (at 37° C.), while the pooled normal plasma (PNP) was warmed in the reagent well (at 37° C.). For these studies, 100 μL of the sample (typically consisting of fibrinogen, calcium and activator) was combined within a cuvette with 100 μL of pooled normal plasma and the rate of clot formation was measured.

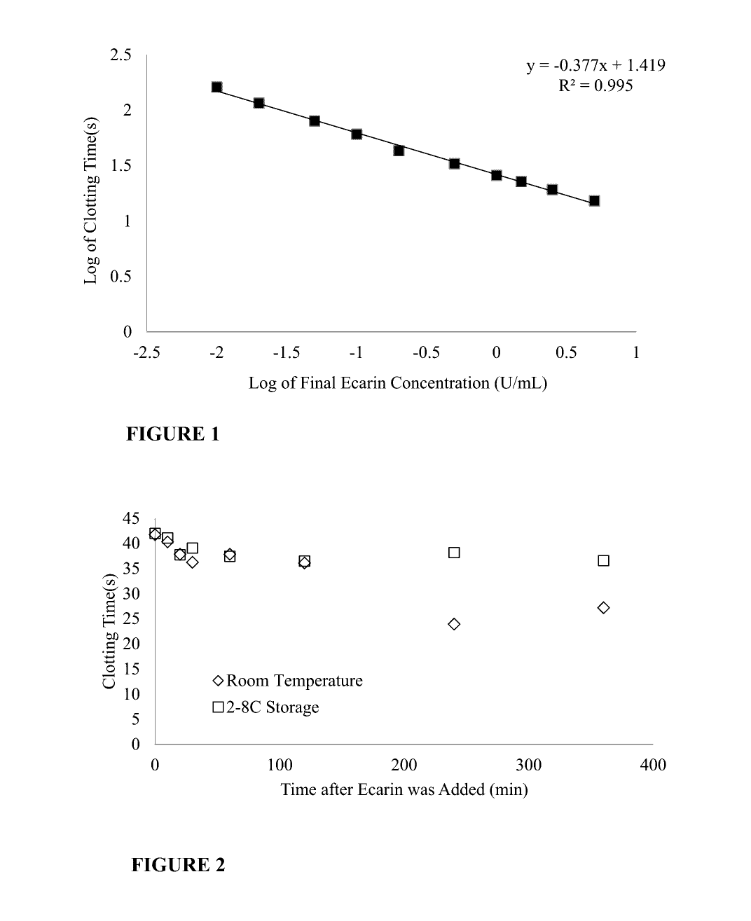

As shown in FIG. 1, Ecarin significantly reduced the clotting time of fibrinogen/calcium with plasma, even at the lowest concentration. There was a clear dose response—increase in Ecarin concentration resulted in reduction of clotting time. In log scale, the dose response was quite linear, with an R squared value of 0.9952.

TABLE 2
Ecarin Dose Response

| Final Ecarin Concentration (U/ml) | Average Clotting Time (s) |
|---|---|
| 5.0 | 15.2 |
| 2.5 | 19.2 |
| 1.5 | 22.7 |
| 1.0 | 25.8 |
| 0.5 | 32.9 |
| 0.2 | 43.0 |
| 0.1 | 60.7 |
| 0.05 | 79.9 |
| 0.02 | 116.0 |
| 0.01 | 161.9 |
| 0 | 700.7 |

### 2B: Stability Study

The purpose of this study was to determine the stability of Ecarin with fibrinogen and calcium. Fibrinogen, calcium, and Ecarin were combined to achieve of 10 mg/mL, 10 mM and 1 U/mL final concentrations, respectively, and incubated at different times and temperatures. The clotting time with plasma was measured using the analyzer at various times after TO (the time the Ecarin was added to the fibrinogen/calcium).

The change in the clotting over time was used to assess the stability of the Ecarin/fibrinogen/calcium mixture. The stability was measured when the solution was stored either at room temperature or at a temperature of 2° C.-8° C. (in the refrigerator).

As can be seen in Table 3 below and in FIG. 2, the clotting time varied slightly over the course of the study but it generally remained with a range of 25-42 seconds.

TABLE 3
Ecarin Stability

| Time After Ecarin | Average Clotting Time | |
|---|---|---|
| Addition to Fibrinogen | Stored at room Temp. | Stored at 2-8° C. |
| 0 min | 41.8 | 42.0 |
| 10 min | 40.3 | 41.1 |
| 20 min | 37.9 | 37.8 |
| 30 min | 36.3 | 39.1 |
| 1 hr | 37.9 | 37.5 |
| 2 hr | 36.1 | 36.5 |
| 4 hr | 24.0 | 38.2 |
| 6 hr | 27.2 | 36.6 |
| 1 day | 29.4 | 29.4 |
| 2 day | 29.4 | 26.7 |
| 5 day | 33.8 | 35.4 |

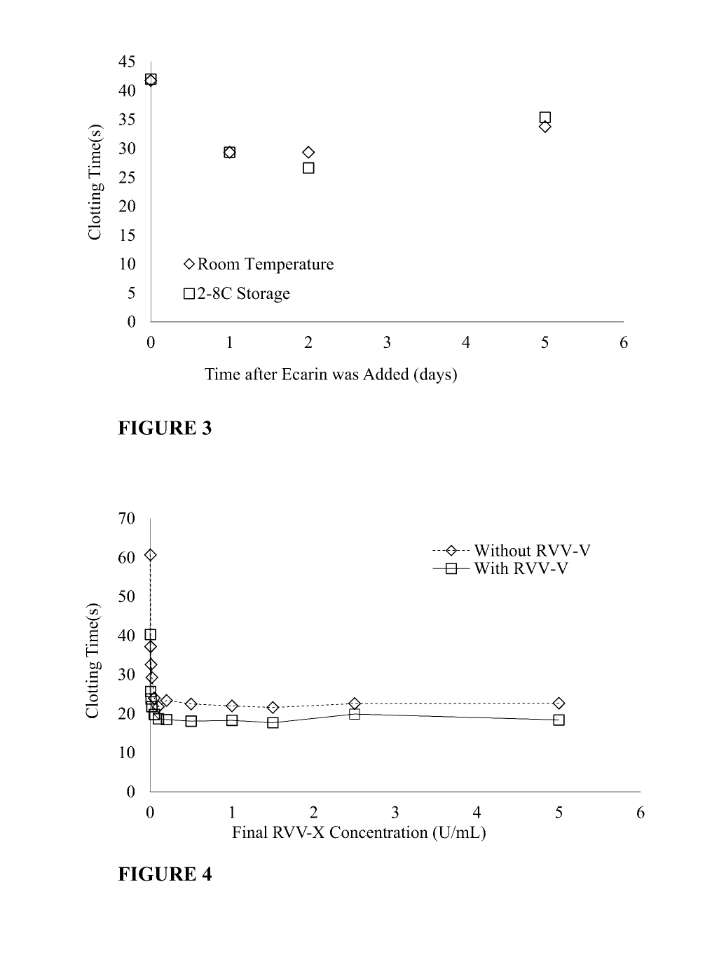

Further, FIG. 3 shows the long term stability of Ecarin in solution with fibrinogen and calcium at time points during a period of up to 5 days based on the clotting time data.

### 2C: Ecarin Calcium Dependence Study.

The objective of this study was to determine the calcium-dependence of Ecarin in reducing the clotting time of fibrinogen and plasma. Ecarin was added to fibrinogen with and without calcium and then the clotting time with plasma was measured.

As shown in Table 4, the fibrinogen/calcium/Ecarin mixture clotted at approximately the same rate as the control sample, i.e. fibrinogen and Ecarin without calcium, thus the effect of Ecarin on clotting time was determined to be not calcium dependent.

TABLE 4
Ecarin Calcium Dependence Data

| Final Ecarin concentration | Calcium Concentration | Average |
|---|---|---|
| (U/ml) | (mM) | Clotting Time (s) |
| 1 | 10 | 34.3 |
| 1 | 0 | 35.4 |

### Example 3: RVV-X and RVV-V Effect on Clotting

### 3A: RVV-X and RVV-V Clotting Time

The objective of this study was to determine the clotting time of fibrinogen supplemented with RVV-X and/or RVV-V, and calcium when combined with plasma. Several concentrations of RVV-X with and without RVV-V were tested to determine the dose response. Specifically, concentrations of RVV-X ranged from 0-5 units per mL, and the concentration of RVV-V was 10 units per mL on occasions when it was added.

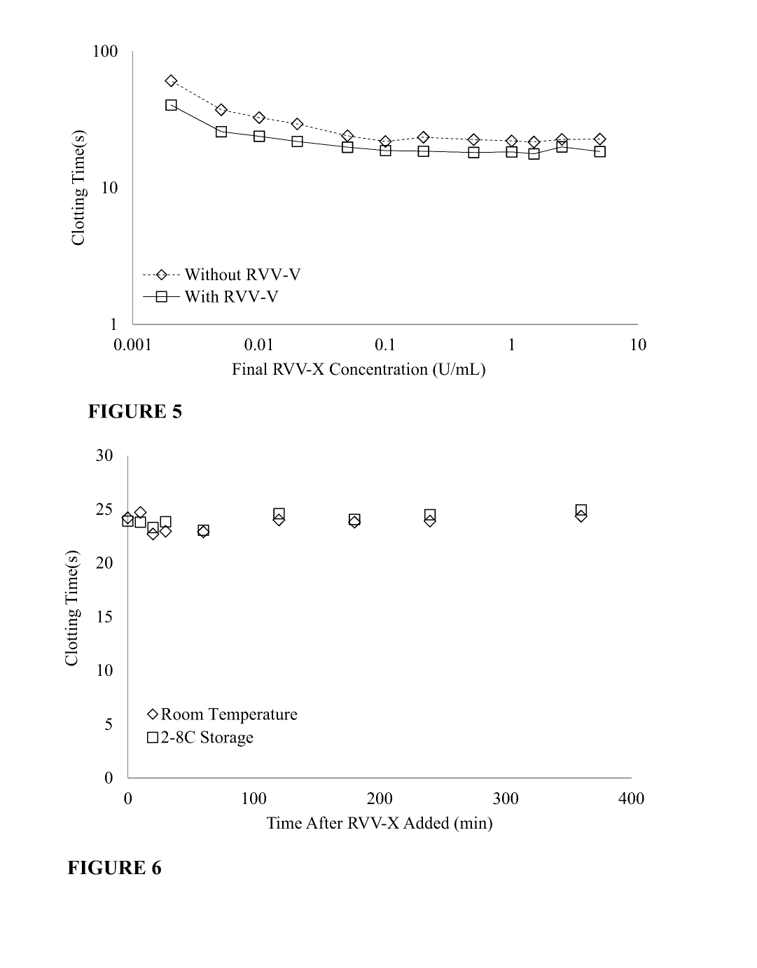

As shown in Table 5, FIG. 4 and FIG. 5, RVV-X significantly reduced clotting time of fibrinogen and calcium with plasma, but there was no clear dose response at concentration greater than 0.1 U/ml, as the clotting times remained about the same until the RVV-X was at lower concentrations. Without being bound by theory, this may be a result of involvement in other factors in the reaction that may be rate-limiting, perhaps phospholipid levels.

When RVV-V was added in the presence of RVV-X, the clotting time was slightly reduced. RVV-V significantly reduced clotting time when there was no RVV-X present, from 520.1 seconds to 141.3 seconds. This indicated that RVV-V is playing a critical role in activation and can reduce clotting times if present in the formulation.

TABLE 5
RVV Dose Response

| Final RVV-X | Average Clotting Time (s) | |
|---|---|---|
| concentration | Without RVV-V | With RVV-V* |
| 5 | 22.7 | 18.4 |
| 2.5 | 22.6 | 19.9 |
| 1.5 | 21.6 | 17.7 |
| 1.0 | 22.0 | 18.3 |
| 0.5 | 22.5 | 18.1 |
| 0.2 | 23.4 | 18.5 |
| 0.1 | 21.8 | 18.7 |
| 0.05 | 24.0 | 19.8 |
| 0.02 | 29.3 | 21.8 |
| 0.01 | 32.6 | 23.8 |
| 0.005 | 37.2 | 25.7 |
| 0.002 | 60.7 | 40.3 |
| 0 | 520.1 | 141.3 |

*The RVV-V final concentration was 10 U/ml.

### 3B: RVV-X Stability Study

The purpose of this study was to determine the stability of RVV-X with fibrinogen and calcium. Fibrinogen, calcium, and RVV-X were combined (to achieve final concentrations of 10 mg/mL, 10 mM and 1 unit/mL, respectively) and then the clotting time with plasma was measured at various times after TO (the time the RVV-X was added to the fibrinogen/calcium). The change in the clotting time over a period of 6 days was used to assess the stability of the RVV-X/fibrinogen/calcium mixture. The stability was measured when the solution was stored either at room temperature and at a temperature of 2° C.-8° C. (in the refrigerator).

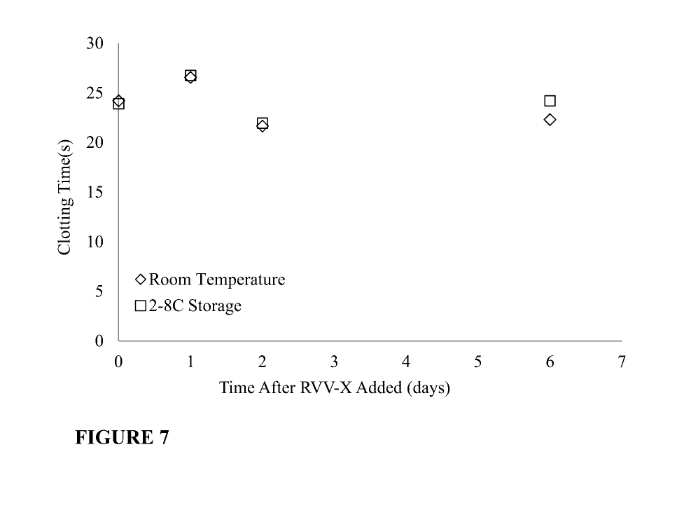

As shown in Table 6, FIG. 6 and FIG. 7, the clotting times remained in a narrow range of 21-27 seconds over the course of the study. There was not a substantial difference between samples stored at room temperature or in the refrigerator at 2-8° C.

The stability of RVV-X in solution with fibrinogen and calcium was demonstrated at time points during a period of 6 days based on the clotting time data presented here.

TABLE 6
RVV-X Stability Data

| Time After RVV-X | Average Clotting Time | |
|---|---|---|
| Addition to Fibrinogen | Stored at room Temp. | Stored at 2-8° C. |
| 0 min | 24.2 | 23.9 |
| 10 min | 24.7 | 23.8 |
| 20 min | 22.7 | 23.3 |
| 30 min | 23.0 | 23.9 |
| 1 hr | 22.9 | 23.1 |
| 2 hr | 24.0 | 24.6 |
| 3 hr | 23.8 | 24.1 |
| 4 hr | 23.9 | 24.5 |
| 6 hr | 24.4 | 25.0 |
| 1 day | 26.6 | 26.8 |
| 2 day | 21.7 | 22.0 |
| 6 day | 22.3 | 24.2 |

### 3C: RVV-X Calcium Dependence

The objective of this study was to determine the calcium-dependence of RVV-X in reducing the clotting time of fibrinogen and plasma. RVV-X was added in an amount of 1 U/mL to fibrinogen (10 mg/mL) with and without 10 mM calcium and then the clotting time with plasma was measured.

As shown in Table 7, the fibrinogen/calcium/RVV-X mixture clotted around the same time as expected based on the dose response study, namely, around 20 seconds. However, the control sample which had no calcium did not clot in the coagulation analyzer, indicating the activity of RVV-X is calcium dependent.

TABLE 7
RVV-X Calcium Dependence

| Final RVV-X concentration | Calcium Concentration | Average |
|---|---|---|
| (U/ml) | (mM) | Clotting Time (s) |
| 1 | 10 | 20.6 |
| 1 | 0 | 999 |

### Example 4: Effect of Freezing and Thawing

The objective of this study was to determine if freezing is a viable method of storage for formulations with RVV-X and/or Ecarin as the exogenous activators. To this end, fibrinogen (10 mg/mL) solutions with calcium (10 mM) and activator (either 1 U/mL RVV-X or 1 U/mL of Ecarin) were prepared and tested with pooled plasma to determine clotting times. A portion of those samples was frozen overnight at −70° C., thawed the next day and clotting times with plasma were again determined.

As shown in Table 8, there was no significant change in the clotting time before and after freezing, so freezing is a viable method for storing the solutions.

TABLE 8
Effect of freezing and thawing

| Exogenous Zymogen | Initial | Clotting Time after |
|---|---|---|
| Activator | Clotting Time (s) | Freeze-Thaw (s) |
| Ecarin | 31.0 | 30.9 |
| RVV-X | 21.3 | 21.2 |

1. A sealant formulation comprising a blend containing fibrinogen and an activator of a member of a mammalian blood clotting cascade, wherein said activator is exogenous to said mammalian cascade and said member is upstream to fibrinogen in the mammalian's blood clotting cascade.

2. The sealant formulation of claim 1, wherein said blend is in dry form or is in a form of a liquid blend.

3. The sealant formulation of claim 1, wherein said blend comprises fibrinogen and the activator at a molar ratio of between 70,000:1 and 25:1.

4. The sealant formulation of claim 1, wherein said member of the blood clotting cascade is a zymogen.

5. The sealant formulation of claim 1, wherein said activator is of non-human source.

6. The sealant formulation of claim 1, wherein said activator comprises venom, a venom component or an analog of a venom component.

7. The sealant formulation of claim 6, wherein the venom component is selected from the group consisting of Ecarin, Russell's Viper venom X factor (RVV-X), Russell's Viper venom V factor (RVV-V), Noscarin, Oscutarin, Trocarin, Convulxin, Botrocetin and any combination of said venom component.

8. The sealant formulation of claim 1, wherein the activator is a proteolytic enzyme active upstream to said member of the clotting cascade or a recombinant form of said proteolytic enzyme.

9. The sealant formulation of claim 1, wherein the activator is active on prothrombin.

10. The sealant formulation of claim 1, wherein the activator is active on factor X.

11. The sealant formulation of claim 1, wherein the activator is active on factor V.

12. The sealant formulation of claim 1, being free of thrombin or a functional analog of thrombin.

13. The sealant formulation of claim 1, comprising calcium.

14. The sealant formulation of claim 1, wherein said fibrinogen is obtained by cryo-precipitation.

15. The sealant formulation of claim 1, being stable at room temperature for at least 5 days and/or being stable at 2° C.-8° C. for at least 5 days.

16. An applicator comprising (i) a barrel holding a sealant formulation comprising a blend containing fibrinogen and an activator of a member of a mammalian blood clotting cascade, wherein said activator is exogenous to said mammalian cascade and said member is upstream to fibrinogen in the mammalian blood clotting cascade; and (ii) a re-sealable opening for delivery therethrough of the sealant formulation.

17. The applicator of claim 16, being a form of a syringe holding said sealant formulation in liquid form.

18. A wound dressing comprising a support matrix holding a sealant formulation, the sealant formulation comprises a blend containing fibrinogen and an activator of a member of a mammalian blood clotting cascade, wherein said activator is exogenous to said mammalian and said member is upstream to fibrinogen in the mammalian blood clotting cascade.

19. A method for promoting the formation of a fibrin clot, the method comprises contacting blood with a sealant formulation comprising a blend containing fibrinogen and an activator of a member of a mammalian blood clotting cascade, wherein said activator is exogenous to said mammalian cascade and said member is upstream to fibrinogen in the blood clotting cascade.

20. The method of claim 19, wherein said sealant formulation is in liquid form.

21. A method of treating a bleeding wound in a subject in need thereof, the method comprises applying onto at least a portion of said wound an amount of a sealant formulation in a form of a blend containing fibrinogen and an activator of a member of a mammalian blood clotting cascade, wherein said activator is exogenous to said mammalian cascade and said member is upstream to fibrinogen in the mammalian blood clotting cascade, the amount of said sealant formulation is effective to promote clotting in said wound when brought into contact with said wound.

22. A kit comprising:
a. a sealant formulation comprising a blend containing fibrinogen and an activator of a member of a mammalian blood clotting cascade, wherein said activator is exogenous to said mammalian cascade and said member is upstream to fibrinogen in the mammalian blood clotting cascade;
b. instructions for use of the sealant formulation to promote clotting in a wound, said instructions comprises applying the sealant formulation onto at least a portion of said wound.

23. The kit of claim 22, wherein said instructions comprise providing said sealant formulation in liquid form.

24. The kit of claim 22, comprising a sealant applicator configured to facilitate said applying of the sealant formulation onto and/or into the wound.
